(19) United States
(12) Patent Application Publication
(10) Pub. No.: US 2023/0019077 A1
(43) Pub. Date: Jan. 19, 2023

(54) STATIC ELECTRICITY RECHARGING DEVICE FOR A USED MASK

(71) Applicant: RF MATE CO., LTD., Siheung-si (KR)

(72) Inventor: Sang-ki WOO, Siheung-si (KR)

(21) Appl. No.: 17/642,742

(22) PCT Filed: Apr. 7, 2021
(86) PCT No.: PCT/KR2021/004338

§ 371 Date: Mar. 14, 2022

(30) Foreign Application Priority Data
Apr. 8, 2020 (KR) 10-2020-0042705

(51) Int. Cl. H02N 1/08; A41D 13/11

(52) U.S. Cl. CPC H02N 1/08; A41D 13/11

(57) ABSTRACT

A mask has to possess sufficient electrostatic charges in order to maintain a filtering effect of the mask using static electricity. However, an electrostatic property naturally decreases as an amount of the electrostatic charges decreases when contacting a human body or exposed to the air. Resultantly, as an initial electrostatic filtering effect gradually decreases, an initially intended function of the mask is not implemented.

The present invention relates to a static electricity recharging device for a mask, which allows the mask to be reused by recharging reduced or removed electrostatic charges in the mask.

### TECHNICAL FIELD

The present invention relates to a static electricity recharging device for a used mask. The mask is required to possess sufficient electrostatic charges in order to maintain a filtering effect of the mask using static electricity. However, an electrostatic property naturally decreases as an amount of the electrostatic charges decreases when contacting a human body or exposed to the air. Resultantly, as an initial electrostatic filtering effect gradually decreases, an initially intended function of the mask is not implemented.

The present invention also relates to a device capable of recharging reduced or removed electrostatic charges in the mask and disinfecting bacteria attached while being used in order to reuse a mask that is not reusable in the related art.

### BACKGROUND ART

Although various masks are used to prevent fine dust inhalation or virus infection, an electrostatic mask using an electrostatic filter is most widely used among the various masks.

In case of the electrostatic mask, a raw material typically has own electrostatic charges by using high voltage electricity in a manufacturing process of the raw material. However, when the electrostatic mask is exposed to the air or contacts all sorts of charged materials, an amount of initial charges is naturally and gradually reduced.

Particularly, when the mask is used for a long time or cleaned, the amount of the electrostatic charges is remarkably reduced to cause degradation in own filtering performance of the electrostatic mask. Thus, basically, the mask may not be used after a usage lifespan thereof, and a new mask should be used.

When electrostatic voltages of various electrostatic masks that are currently selling on the market are measured immediately after opening packages, the electrostatic voltages have different intensities in a range from 2,000V to 3,000V or a range from 9,000V to 10,000V. Even a defective mask that has almost no static electricity therein is distributed in the market.

Also, even a mask maintaining electrostatic charges at a predetermined level or more may minimally have 20% or less from an initial level when five to six hours elapses after opening the package although the mask is not worn. When the mask is substantially worn, a reduction speed of the electrostatic charges is much faster due to a human body.

That is, when a typical static electricity mask is used for a long time or repeatedly used, the mask may be degraded in own filtering performance as static electricity is discharged, and a user may have the risk of infection when exposed to viruses because the performance of the mask is maintained for a short time.

Also, bacteria such as Escherichia coli may be grown on a surface of the mask due to the reuse to cause a secondary health problem.

### DISCLOSURE OF THE INVENTION

### Technical Problem

The present invention provides a static electricity recharging device for a mask, which allows a used electrostatic mask to be used for a long time and/or reusable by recharging static electricity of the used mask in which an electrostatic filtering effect is reduced due to usage or reduced due to mask cleaning or mask disinfection and simultaneously disinfecting viruses or bacteria attached to the mask using ions or ozone in order to solve the above-described problems of the typical electrostatic mask.

### Technical Solution

Embodiments of the present invention provides a static electricity recharging device for a mask, including: a housing; electrodes disposed in the housing, spaced a predetermined distance from each other while facing each other so that a mask is inserted therebetween, and having a shape covering at least a portion of a filter of an electrostatic mask; and a voltage adjustment unit configured to adjust a voltage applied to the two electrodes from a power. Here, static electricity is charged to the filter of the mask by an electric field formed as the voltage is applied to the two electrodes through the power.

Also, the housing may include: a first housing; a second housing separated from the first housing; and a connection member configured to connect the first housing and the second housing in a rotatable manner. Here, one of the electrodes facing each other may be fixed inside the first housing, and the other of the electrodes facing each other may be fixed inside the second housing.

Also, a dielectric layer may be formed on a surface of each of the electrodes facing each other. Here, the dielectric layer may be made of a material having an electrification rank higher than the electrostatic mask.

The dielectric layer formed on the surface of each of the electrodes facing each other may be made of a material having a different dielectric property. Here, all of the two dielectric layers may be made of the material having the electrification rank higher than the electrostatic mask. One of the dielectric layers having different dielectric properties may be made of the material having the electrification rank higher than the electrostatic mask, and the other may be made of a material having the electrification rank lower than the electrostatic mask.

The static electricity recharging device may further include a switching element configured to recharge a positive (+) charge or a negative (−) charge for matching the kind of a charge formed on the electrostatic mask with a usage purpose.

Also, each of the electrodes facing each other may have a planar structure or a three-dimensional structure that is equal or similar to a shape of the electrostatic mask. Also, the power may include a secondary battery that is rechargeable after used.

### Advantageous Effects

The device according to the present invention may recharge the static electricity of the mask having the electrostatic filtering performance degraded due to long time use and continuously maintain the filtering effect at the proper level to extend the usage time of the electrostatic mask.

Also, the device according to the present invention may simultaneously obtain the effect of disinfecting the viruses or bacteria attached to the mask by using the ions and ozone generated during the static electricity recharging to reuse the mask in the more hygienic manner.

Also, the device according to the present invention may reuse the mask by recharging the static electricity, at the level similar to the initial state, of the mask from which the static electricity is removed after cleaning or disinfecting the electrostatic mask in which the contaminant material such as fine dusts or bacteria are accumulated.

### BRIEF DESCRIPTION OF THE DRAWINGS

FIG. 1 is a perspective view illustrating a static electricity recharging device for a used mask according to an embodiment of the present invention.

FIG. 2 is a perspective view illustrating an opened state of the static electricity recharging device for the used mask according to an embodiment of the present invention.

FIG. 3 is a bottom perspective view illustrating the static electricity recharging device for the used mask according to an embodiment of the present invention.

FIG. 4 is a perspective view illustrating a state in which components such as a power or an electrode, which configure the static electricity recharging device for the used mask, are separated from a housing according to an embodiment of the present invention.

FIG. 5 is a configuration view illustrating a system of the static electricity recharging device for the used mask according to an embodiment of the present invention.

FIG. 6 is a usage state view illustrating the static electricity recharging device for the used mask according to an embodiment of the present invention.

### MODE FOR CARRYING OUT THE INVENTION

Hereinafter, preferred embodiments of the present invention will be described in detail with reference to the accompanying drawings.

Terms used in the following description and scopes of claims are not limited to terms that have been in dictionaries, and are used only for explain specific exemplary embodiments while not limiting the present invention. The invention may, however, be embodied in many different forms and should not be construed as being limited to the embodiments set forth herein; rather, these embodiments are provided so that this disclosure will be thorough and complete, and will fully convey the concept of the invention to those skilled in the art.

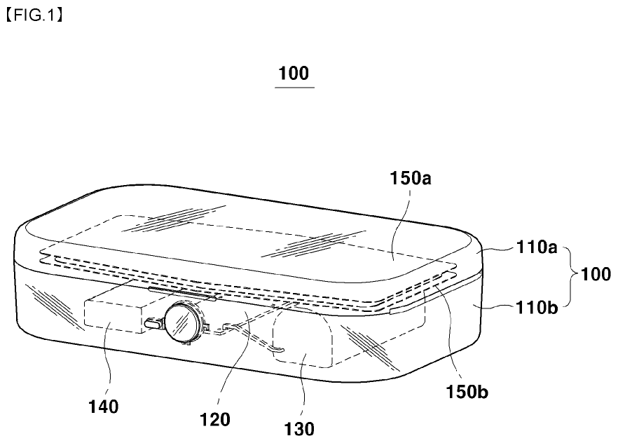

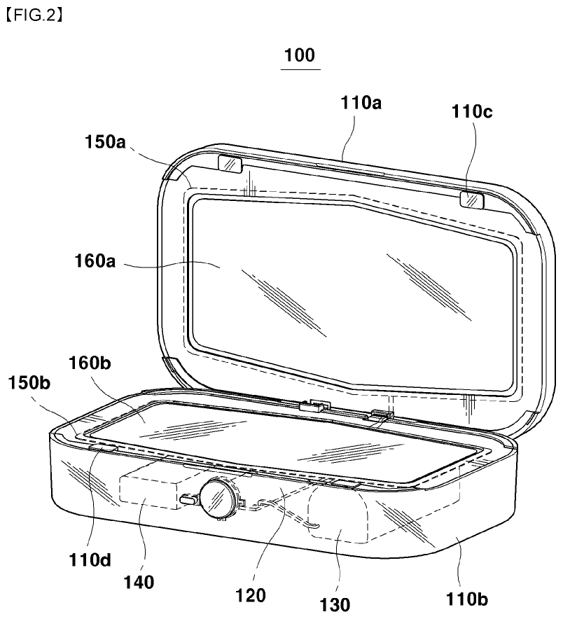

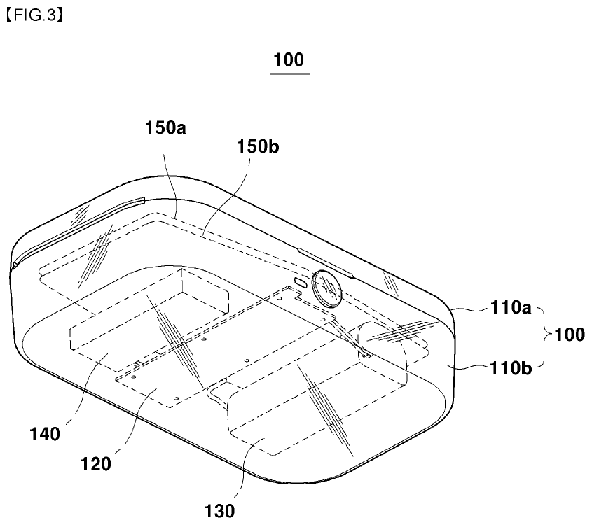

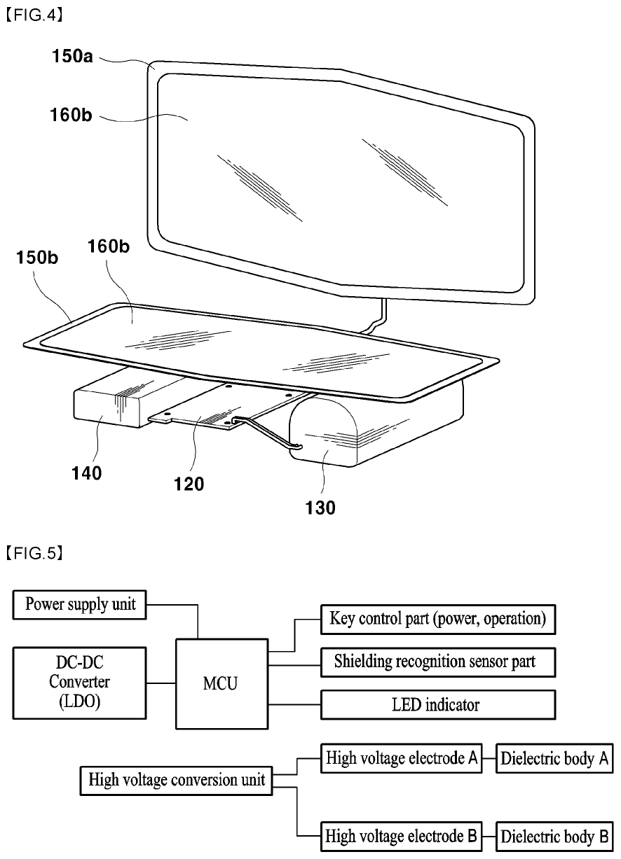

FIG. 1 is a perspective view illustrating a static electricity recharging device for a used mask according to an embodiment of the present invention, FIG. 2 is a perspective view illustrating an opened state of the static electricity recharging device for the used mask according to an embodiment of the present invention, FIG. 3 is a bottom perspective view illustrating the static electricity recharging device for the used mask according to an embodiment of the present invention, and FIG. 4 is a perspective view illustrating a state in which components such as a power or an electrode, which configure the static electricity recharging device for the used mask, are separated from a housing according to an embodiment of the present invention.

As illustrated in FIGS. 1 to 4, a static electricity recharging device 100 according to the present invention includes a housing 110, a control unit 120, a high voltage conversion circuit unit 130, a power supply unit 140, an electrode 150a and 150b, a dielectric body 160a and 160b, and a switching unit 170 for turning a power on or off.

The housing 110 includes an upper housing 110a having an approximately cuboid shape having an opened one surface and an inner accommodation space and a lower housing 110b separated from the upper housing 110a. Although not shown, the housing 110 may be opened and closed as the upper housing 110a and the lower housing 110b are connected through a rotating connection member such as a hinge. A fixing unit 110c and 110d capable of fixing a closed state of both housings may be formed on both ends of the upper housing 110a and the lower housing 110b. Although the rotating connection member is used in an embodiment of the present invention, the both housings may be fastened by a vertical or horizontal slide shielding method or by using a simple fastening unit without motion constraint (e.g., a method of press-fitting the upper housing 110a to an outer circumferential portion of the lower housing 110b in an interference fit manner.

The upper housing 110a has a cuboid shape having an opened portion at one side thereof and an inner space, and a fixing unit for fixing the electrode 150a is installed in the upper housing 110a. Also, the lower housing 110b also has a cuboid shape having an opened portion at one side thereof and an inner space, and a fixing unit for fixing the electrode 150b is installed in the lower housing 110b. Also, the control unit 120, the high voltage conversion circuit unit 130, and the power supply unit 140 are disposed at one side of the lower housing 110b.

The control unit 120 generates a control signal for controlling the overall device. For example, micro controller unit (MCU) may be used as the control unit 120. In this case, as illustrated in FIG. 5, the control unit 120 may include: a key control part for controlling a key that is a manipulation part installed on the device; a shielding recognition sensor part for checking whether the upper and lower housings 110a and 110b are opened or closed to determine an operation state of the device; a LED indicator part for displaying all sorts of states to a user; and a DC-DC converter for controlling a current supplied to the electrode. Although the control unit 120 is configured as a system illustrated in FIG. 5 in an embodiment of the present invention, it is obvious that another function may be added as necessary, or a function that is not essential for operating the device among existing functions may be selectively excluded.

The high voltage conversion circuit unit 130 converts a voltage supplied through the DC-DC converter into a high voltage at a level of recharging static electricity to an electrostatic mask. The converted level of the voltage may be controlled differently according to a level of an electrostatic voltage required for a mask filter.

The power supply unit 140 supplies a power for operating the device. Since the device of the present invention may operate for a sufficient time and have high portability in an embodiment of the present invention, a battery (preferably, a rechargeable secondary battery) is used as the power. However, the embodiment of the present invention is not limited thereto. For example, a general power supplied from the outside may be used.

As illustrated in FIGS. 2 and 4, the electrode 150a and 150b may include a first electrode 150a and a second electrode 150b, which face each other and each have a plate shape. The two electrodes may each have a preferred shape corresponding to a shape of a mask inserted between the two electrodes. Also, a distance between the two electrodes may be properly adjusted in consideration of the mask inserted therebetween and a recharging efficiency. Also, each of the first electrode 150a and the second electrode 150b may be formed as a single electrode or an assembly of a plurality of electrodes.

Although the plate shaped electrode is manufactured in an embodiment of the present invention, a net-shaped electrode, e.g., a mesh-shaped electrode, may be used.

Although the dielectric body 160a and 160b is not an essential component for recharging static electricity to the electrostatic mask, the dielectric body 160a and 160b serves to effectively charge static electricity to the used mask.

A first dielectric body 160a is disposed on a surface facing the mask in the first electrode 150a, and a second dielectric body 160b is disposed on a surface facing the mask in the second electrode 150b.

Recharging may be well performed when each of the two dielectric bodies 160a and 160b has an electrification rank higher than an electrification material of an electrostatic filter of the rechargeable mask, or one of the two has a higher electrification rank, and the other has a lower electrification rank lower than the electrification material of the electrostatic filter of the rechargeable mask

Thereafter, an operation of the static electricity recharging device for the used mask according to an embodiment of the present invention will be described.

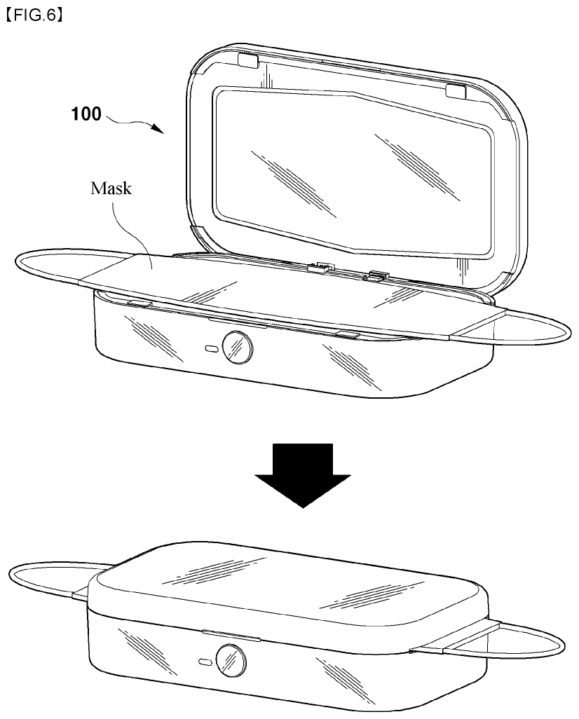

FIG. 6 is a usage state view illustrating the static electricity recharging device for the used mask according to an embodiment of the present invention. As illustrated in FIG. 6, the mask for static electricity recharging is put in the device by opening the first housing 110a, putting the mask, which is degraded in filter performance due to long time use or cleaned or disinfected as many contaminants are attached thereto, on the second electrode 150b, and then closing the first housing 110a.

When the first housing 110a is closed, the shielding recognition sensor part senses as an operational state.

Thereafter, when the user pushes a switch 170, the control unit 120 supplies the power to the DC-DC converter, which applies a high voltage capable of generating static electricity in the filter to the first and second electrodes 150a and 150b through the high voltage conversion circuit unit 130. The high voltage applied to the electrode induces static electricity to the mask through the first and second dielectric bodies 160a and 160b. Here, an electric field is formed between both electrodes to which the high voltage is applied, the formed electric field causes dielectric polarization of an air layer, and the dielectric polarization generates ions and ozone. Since the generated ions and ozone kill viruses and bacteria attached to the mask, a disinfection treatment effect may be simultaneously obtained with the static electricity recharging. When a predetermined time elapses, the control unit 120 stops applying the power, and the LED indicator part displays a mark indicating that the static electricity recharging is completed.

Although the static electricity recharging is further effectively performed by using the first and second dielectric bodies 160a and 160b in an embodiment of the present invention, the static electricity recharging may be performed without the dielectric bodies. Thus, the embodiment of the present invention is not limited to the dielectric bodies.

### DESCRIPTION OF REFERENCE NUMERAL

100: Static electricity recharging device
110: Housing
120: Control unit
130: High voltage conversion circuit unit
140: Power supply unit-Battery
150a, 150b: Electrode A, Electrode B
160a, 160b: Dielectric body A, Dielectric body B
170: Switch

1. A static electricity recharging device for a mask, comprising:
a housing;
electrodes disposed in the housing, spaced a predetermined distance from each other while facing each other so that a mask is inserted therebetween, and having a shape covering at least a portion of a filter of an electrostatic mask; and
a voltage adjustment unit configured to adjust a voltage applied to the two electrodes from a power,
wherein static electricity is charged to the filter of the mask by an electric field formed as the voltage is applied to the two electrodes through the power.

2. The static electricity recharging device of claim 1, wherein the housing comprises:
a first housing;
a second housing separated from the first housing; and
a connection member configured to connect the first housing and the second housing in a rotatable manner,
wherein one of the electrodes facing each other is fixed inside the first housing, and the other of the electrodes facing each other is fixed inside the second housing.

3. The static electricity recharging device of claim 1, wherein a dielectric layer is formed on a surface of each of the electrodes facing each other.

4. The static electricity recharging device of claim 3, wherein the dielectric layer is made of a dielectric material having an electrification rank higher than the electrostatic mask.

5. The static electricity recharging device of claim 3, wherein the dielectric layer formed on the surface of each of the electrodes facing each other is made of a material having a different dielectric property.

6. The static electricity recharging device of claim 1, further comprising a switching element configured to recharge a positive (+) charge or a negative (−) charge for matching the kind of a charge formed on the electrostatic mask with a usage purpose.

7. The static electricity recharging device of claim 1, wherein each of the electrodes facing each other has a planar structure or a three-dimensional structure that is equal or similar to a shape of the electrostatic mask.

8. The static electricity recharging device of claim 1, wherein the power comprises a secondary battery that is rechargeable after used.
